(19) United States
(12) Patent Application Publication
(10) Pub. No.: US 2011/0275915 A1
(43) Pub. Date: Nov. 10, 2011

(54) Arm and wrist cuffs and pulse oximeter clip with conductive material for electrodes on small medical home monitors

(76) Inventor: Dean O. Allgeyer, Los Angeles, CA (US)

(21) Appl. No.: 13/066,593

(22) Filed: Apr. 19, 2011

Related U.S. Application Data
(60) Provisional application No. 61/395,234, filed on May 10, 2010.

(51) Int. Cl. A61B 5/02; A61B 5/04

(52) U.S. Cl. 600/324; 600/372; 600/509

(57) ABSTRACT

This invention is an improvement to medical devices used for home and remote monitoring. The improvements include a coated fabric electrode used for arm and wristbands and for pulse oximeter clips. The electrode is comprised of the hook portion of hook and loop material that is coated with material made from a noble metal such as silver.

This application is based on Provisional Application No. 61/395,234, filed May 10, 2010.

### FIELD OF THE INVENTION

This invention relates to arm and wrist cuffs and a pulse oximeter clip with conductive material functioning as electrodes for medical devices. In particular it relates to small monitoring devices that patients can use at home to monitor important information such as vital signs, which can also be used by physicians for remote patient monitoring.

### BACKGROUND OF THE INVENTION

With the continuing focus on the cost of medical care, and in particular with the efforts to avoid unnecessary physician and hospital visits, there is a need to facilitate patient care at home while also simplifying the patient's or caregiver's task of monitoring the patient's symptoms or vital signs. Europe appears to already be ahead of the United States in the home monitoring of patients, although it is expected that continually increasing costs will force the United States to follow suit. The basis of the cost saving is the efficient and productive delivery of goods and services, which is always desirable.

Some basic devices have existed for a decade or more, such as small automatic blood pressure monitors that require nothing more than the patient correctly put on the arm cuff and push a start button. One such device is the model HEM-712C with IntelliSense sold by Omron Healthcare, Inc. In an attempt to simply the cuff even more, some companies have created small cuffs that fit a patient's wrist and have adapted their algorithms for calculating blood pressure to account for the differences sensors obtain when reading the pressure at the wrist rather than the arm. Philips makes a number of devices for remote, in-home monitoring, including a blood pressure monitor, an ECG rhythm strip recorder, and a pulse oximeter. A Portable Handheld ECG Monitor by ReadMyHeart sells on Amazon for $164.99. Beijing Choice Electronic Tech. Co., Ltd. also makes a small, inexpensive, handheld ECG monitor.

One problem these devices have is that many perform only one function, a situation which can necessitate a patient purchasing three, four, or five different devices, and in some cases compile and provide that information to a nurse or a physician. Philips' collection of blood pressure, pulse oximeter, ECG, and diabetes monitoring devices are a good example. A solution to this problem appears in U.S. Pat. No. 7,610,085 to Allgeyer, which involves placing electrodes in a blood pressure cuff and in a pulse oximeter mounted on the hand opposite the arm with the blood pressure cuff. This arrangement creates a voltage potential between the cuff and the pulse oximeter, so that one device can provide a blood pressure reading, an oxygen saturation reading, and an ECG rhythm strip.

Another problem with many of the small ECG monitors is the electrodes. The Allgeyer '085 patent recognizes the bactericidal properties of silver and states a preference for a sintered Ag/AgCl coating over wire mesh or carbon matrix to form the electrodes. Because of the well known, non-tarnishing, bactericidal, and conductive properties of silver, there is a natural preference for electrodes that are silver-based. In a similar vein, monitoring devices like the Beijing Choice have solid silver electrodes that a patient presses with his fingertips. Often, however, hand tremors, weakness in the extremities, or similar problems cause an elderly patient to grasp these small devices with uneven and varying pressure, causing distorted readings.

Other efforts have been made to obtain an effective electrical connection with conductive tissue on the surface of the body. One such effort appears in U.S. Pat. No. 6,690,959 to Thompson, which includes metalized nanospikes shaped to penetrate the epidermis. The purpose of the nanospikes is to pierce the stratum corneum—the skin at the very surface of the body—to receive electrical signals in a sufficiently strong manner to sense cardiac depolarization waveforms, i.e., ECGs. These nanospikes, however, are only about 10 microns long so they can avoid contacting nerves or capillaries that could be 200 to 300 microns deep. The '959 patent to Thompson appears to have been intended for use with implantable devices like cardioverters and defibrillators. As a practical matter, it appears that wire mesh, whether coated or uncoated, and other types of electrodes would penetrate more than the 10 microns of Thompson to obtain a better electrical effect.

In an effort to improve the electrode conductivity and signal of the simplified ECG described in the '085 patent to Allgeyer, the present inventor considered a number of solutions. One was a silver-plated nylon conductive fabric with a surface resistivity of less than 0.3 ohms and a nominal thickness of 0.120 mm (0.0045 inches). At times, however, it was difficult to obtain a signal, and when a signal was obtained the ECG monitor often showed significant baseline drift and inconsistent amplitude. It would seem that tightening of the blood pressure cuff with the conductive nylon electrode would have penetrated at least 10 microns into the epidermis, the desired depth of Thompson, leading to the conclusion that Thompson's nanospikes would not be particularly effective as an electrode used in conjunction with the '085 Allgeyer device.

A silver wire mesh electrode was also tried. Patches of silver wire mesh were attached to the inside of the blood pressure cuff. The resulting ECG strips, however, remained somewhat variable and unpredictable. To some extent, this inconsistency arises from the different size arms and fingers of individuals whose vital signs were recorded. Another reason for inconsistency was the quality of the ECG monitors. Conductive glue and conductive thread were tried in an effort to improve readings, but they offered no improvement. Wire mesh was also placed under the silver-plated nylon fabric in an effort to create a more irregular surface that would create more contact if sufficient pressure were applied. This, too, proved unsatisfactory. Recent developments in the field of silver ink have led to increased use of silver ink circuits being printed on a polyester substrate. Polyester substrates, however, proved too stiff to flex properly with an inflating and deflating blood pressure cuff. Moreover, if the substrate was significantly thinner, over time, with continued inflations and deflations, fatigue of the plastic would result in crimping, thus potentially shorting out the conductivity of the electrode in the cuff. In the '085 Allgeyer device, another problem arises. The clothespin-like finger clip sometimes does not have a strong enough spring force to adequately connect the finger with the solid surface of a silver electrode inside the clip. Therefore, it is desirable to have a stronger and more consistent clip, a better electrode to insure adequate contact with the skin, or both.

In view of the state of the art of small, home-monitoring devices, there remains a need for a conductive electrode compatible with arm and wrist cuffs whose size must vary from person to person, and for pulse oximeter clips with limited spring force to secure them to a finger to obtain electrical signals for an ECG rhythm strip.

### SUMMARY OF THE INVENTION

An excellent electrode is the “hook” portion of electrically conductive hook and loop material usually known as Velcro. In the preferred embodiment of a blood pressure arm cuff, a two inch wide strip of the conductive hook material is attached to the inside of the cuff by electrically conductive rivets or compression fittings. These fittings, in turn, are adapted to mechanically and electrically connect with ECG eyelets wired to the monitor that produces the ECG strip.

The conductive hook material can also be used as the electrode on wristbands. In the case of the Allgeyer '085 patent, a wristband can be a blood pressure cuff with an electrode to create the voltage potential for an ECG. On the other hand a patient could have a pulse oximeter clip as described in the '085 patent, the electrode of which could be metal or the conductive hook material. Silver wire mesh and 20-gauge argentinium silver plate have proved effective. If an oxygen saturation reading is not desired, the wristband with the coated hook material is the preferred lead on the side opposite the blood pressure cuff.

Another use of the conductive hook material is with a home ECG monitor. The monitor is connected to two wristbands, each of which has an electrode of the conductive hook material. This particular device is depicted in the attached figures. The cuffs are made of an elasticized fabric and can be secured in a closed position with Velcro placed at the ends of the cuffs. On the inside face of the wristbands a length of the conductive hook material is secured by rivets covered with a conductive coating. The length of the conductive material is slightly shorter than the wristband and is one inch wide. In one preferred embodiment, the elastic and the conductive hook material are attached to each other on three sides of the conductive material to form a pocket. This permits the insertion of a 40 mil thick piece of polystyrene that has been molded in an approximately circular shape so that the wristband will stay on the wrist while a patient is tightening and adjusting the band and securing it around the wrist. ECG eyelets are snapped onto the rivets on the outside of the wristband and are wired to an ECG.

The resulting wristband provides a consistently good ECG signal to a variety of ECG monitors. The amplitude with any monitor remains consistent, and the baseline drift is typically negligible. One of the advantages of the elastic wristband is that it stretches, i.e., tightens, the conductive hook material. This permits the conductive hooks to make better contact with the skin, thus improving the quality of the signal.

### BRIEF DESCRIPTION OF THE DRAWINGS

The features of the invention and the elements characteristic of the invention are described below and set forth in the appended claims. The figures are for illustration purposes only and are not drawn to scale. The invention itself, however, both as to its structure and method of operation, may best be understood by reference to the detailed description which follows, taken in conjunction with the accompanying drawings in which:

FIG. 1 depicts the individual parts of two wristbands, including the conductive hook material that can be used with a home-monitor ECG.

FIG. 2 shows two assembled wristbands.

FIG. 3 is another view of the assembled wristbands that depicts the rivets to which the ECG eyelets connect.

FIG. 4 shows two hands with the wristbands producing an ECG tracing on a computer monitor.

FIG. 5 depicts an arm cuff and pulse oximeter finger clip.

### DETAILED DESCRIPTION OF THE INVENTION

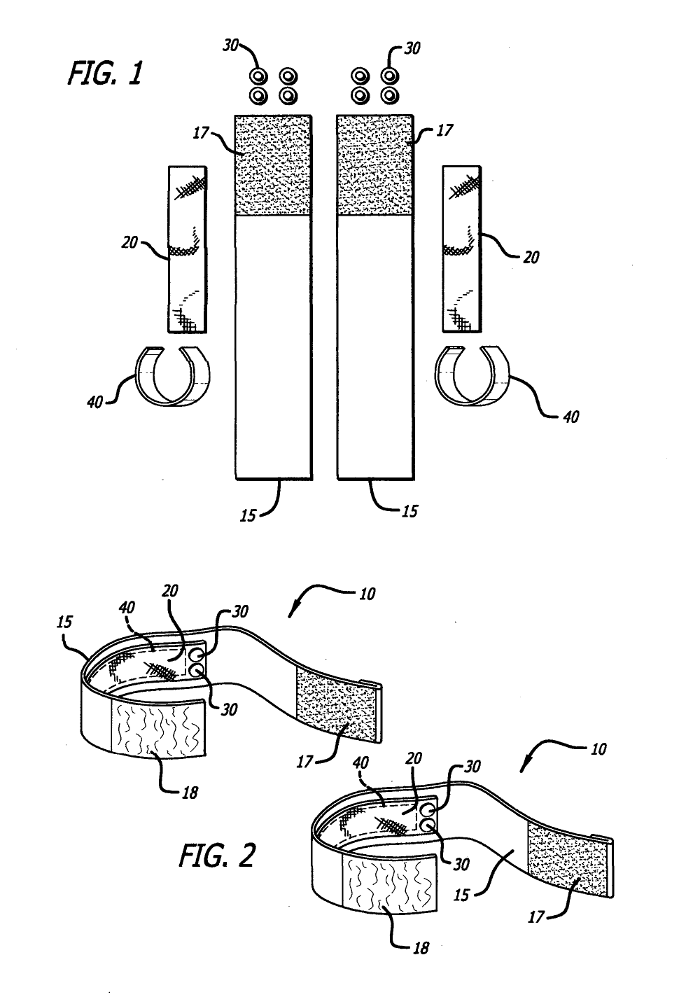

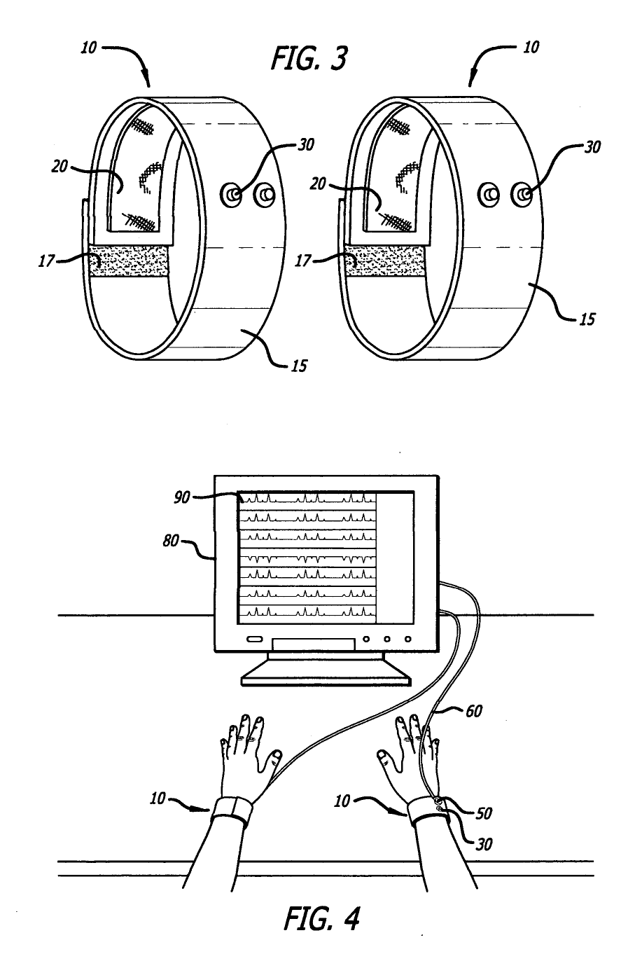

While FIGS. 2 and 3 depict completed wristbands 10, FIG. 1 depicts the unassembled parts. Wristbands 10 are preferably made of elastic bands 15. Here the word “elastic” is used in its general consumer sense as something that can be stretched, like an elastic waistband. Conductive electrodes 20 are made of a coated hook material, as in hook and loop Velcro, which is coated with a conductive material made from noble metals like silver and gold. The electrodes are sewn to the inside of the wristband, with one end of the conductive material left open to form a pocket into which a flexible wrist mount is inserted. Flexible wristband mount 40 is a curved piece of polystyrene that provides shape and structure to wristband 10. The wristband mount 40 also closes around a patient's wrist to keep the wristband on the wrist while the patient secures and tightens the band 10.

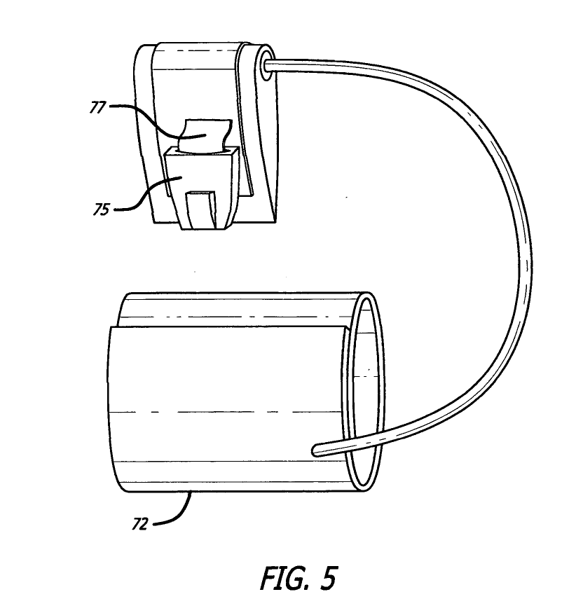

The ends of elastic bands 15 have Velcro, i.e., hook and loop, fasteners to secure the wristband 10 in its final position. As seen in FIG. 2, the interior of wristband 10 has a hook portion 17 that connects to a loop portion 18. In addition, conductive rivets 30 are used to assist in carrying the electrical signal. These rivets comprise a means for connecting the conductive electrode to an ECG monitor. One side of rivet 30 has a nipple-like protrusion (see FIG. 3) that can connect to an ECG eyelet 50 (see FIG. 4). The eyelet 50 is wired to an ECG monitor that produces an ECG tracing 90, such as the one shown on the screen display 80 in FIG. 4. Other means for connecting are well known to those in the art. For example, alligator clips or even mere wires could be directly or indirectly sewn, glued, or soldered to the conductive hook material. Moreover, with time, it is anticipated that technological improvements will create additional means. At this time the rivet and eyelet is the preferred embodiment.

The wristband 10 can also be made of non-elastic material rather than elastic bands 15, but then preferably there should be a piece of elastic material at the end of band 15 to facilitate adjusting and securing the wristband tightly and correctly on the wrist. The electrode 20 is a 25 mm (1 inch) wide strip of the hook portion of the Electrically Conductive Hook & Loop material manufactured by Statex Productions & Vertriebs GmbH of Bremen, Germany. The material is silver plated nylon and is sold in the United States under the trade name Shieldex by VTT/Shieldex Trading USA of Palmyra, N.Y. The material is sold on 25-meter rolls in 25 mm and 50 mm widths. The Shieldex technical data sheet identifies sewing, gluing, and ultrasonic bonding as the methods of attaching Shieldex. All three methods are satisfactory, including the use of conductive threads and glues. For cost purposes, however, the preferable methods of attachment are sewing and riveting, depending on the type of band or cuff with which the Shieldex is being used. In the attached drawings the rivets 30 are compression fittings manufactured or coated with an electrically conductive material.

Surprisingly, the surface resistivity of the electrically conductive hook and loop material is almost five times that of the thin (0.120 mm) silver-plated nylon fabric. Even the resistivity over the closure between the hook and loop is almost three times that of the nylon. Nevertheless, the silver-coated hook material provides better conductivity and more reliable and consistent ECG readings. Presumably this results from the greater stiffness of the hooks, which, unlike the loops, are cut and non-continuous. A substantial portion of the hooks appears to push aside the corium and epidermis enough so that the hooks reach conductive portions of the skin. At the same time, the flexibility and thinness of the hooks do not penetrate so deeply that they cause the patient discomfort.

It should be noted that the preferred embodiment of the electrode is a single piece of conductive material 20. More consistent ECG readings resulted from longer, narrower strips than shorter, wider strips, although the latter configuration is still considered within the scope of the present invention. As noted above, with the wristbands 10, which were approximately 2 inches wide, a 1-inch wide piece of Shieldex was used for conducting hooks 20. In terms of surface area, one strip of conducting hooks 20 represented about ¼ to ⅓ of the surface area of the wristband. See FIG. 1. The 1-inch strip 20 works satisfactorily with wider wristbands, which leads to the conclusion that a narrower strip of conducting material 20 would work well with a narrower wristband.

Two-inch wide strips of conducting loops were used with blood pressure arm cuffs. So, too, were one-inch strips. Two-inch strips produced slightly more consistent amplitudes and less baseline drift, so two-inch strips are preferred with wider cuffs and bands. Nevertheless, narrower strips, like the Shieldex 25 mm strips hooks, worked satisfactorily and should be considered within the scope of the present invention. The conductive hook material is one the most expensive aspects of the invention, so there is a cost-benefit consideration in the width and length of the coated hook material.

A flexible, finger-mounted rubber pulse oximeter provided noticeably more consistent ECG readings than the clothes pin style, because the rubber applied a greater force to the electrode, thus obtaining a better electrical signal. These flexible devices were, it is believed, originated by Hewlett Packard, and they are still sold today by Philips, Beijing Choice, and others. Because of the additional force provided by the rubber clip, the conductive hook material provided as good or better ECG readings than silver mesh and silver plate electrodes. The most important factor in the clip's effectiveness appears to be the circumferential spring force, which comes from the cylindrical or conical shape of the rubber clip. A traditional clothespin-like clip could be equally effective if it had a consistent and strong enough spring force to create contact between the skin and the electrode.

FIG. 5 depicts an arm cuff 72 and the rubber pulse oximeter clip 75 with a small protruding edge 77 of the conductive hook material. Because of the small size of the pulse oximeter clip electrode relative to the cuff electrode, it is easier to mount the electrode on the clip so that the electrode is slightly in tension and the hooks can obtain a good electrical signal.

Variations of the described combinations are possible within the spirit and scope of the invention, which is to be limited only by the following claims and their equivalents.

What is claimed is:

1. In an arm or wrist cuff capable of sensing electrical signals in the skin of a patient, the improvement comprising:
an electrode comprising the hook portion of hook and loop material,
the hook portion being coated with a material made from a noble metal;
the area of the hook material comprising on the order of at least 20 percent of the area of the cuff; and,
the length of the hook material on the order of at least 50 percent of the length of the cuff.

2. The invention of claim 1, wherein the noble metal is silver.

3. The invention of claim 1, wherein the noble metal is gold.

4. The invention of claim 1, further comprising at least one conductively coated rivet for connecting the hook material to an ECG eyelet.

5. A wristband with an electrode for sensing ECG signals that are transmitted from a patient to an ECG monitor, the wristband comprising:
a generally curved, flexible wristband mount;
a wristband mounted on the wristband mount, wherein the wristband and the wristband mount are adapted to adjust to a range of wrist sizes;
an electrically conductive electrode attached to the wristband so that it contacts the patient's skin when secured on the wrist of the patient, the electrode comprising the hook portion of hook and loop material wherein the hook portion has an electrically conductive coating; and
means for connecting the electrode to an ECG.

6. The invention of claim 5, wherein the hook portion is coated with a material made from a noble metal.

7. The invention of claim 6, wherein the noble metal is silver.

8. The invention of claim 6, wherein the hook portion is a unitary piece of material on the order of at least 20 percent of the area of the wristband and at least one half the length of the wristband.

9. The invention of claim 7, wherein the means for attaching comprises metal rivets adaptable to connect with ECG eyelets.

10. The invention of claim 9, wherein the rivets are coated with an electrically conductive material.

11. The invention of claim 5, wherein the wristband is comprised at least in part of a stretchable elastic material to adjust the size and tightness of the wristband.

12. In a finger clip for a pulse oximeter and for creating an ECG rhythm strip, the improvement comprising:
a conductive electrode including the hook portion of hook and loop material, wherein the hook portion is coated with a conductive material made from a noble metal.

13. The invention of claim 12, wherein the noble metal is silver.
